(19) United States
(12) Patent Application Publication
(10) Pub. No.: US 2019/0142399 A1
(43) Pub. Date: May 16, 2019

(54) UTERINE COLLECTION DEVICE AND METHOD

(71) Applicant: Éric Himaya, Gatineau (QC) (CA)

(72) Inventor: Éric Himaya, Gatineau (QC) (CA)

(21) Appl. No.: 15/812,496

(22) Filed: Nov. 14, 2017

(51) Int. Cl. A61B 10/02; A61B 90/00

(52) U.S. Cl. CPC A61B 10/0291; A61B 2090/061; A61B 90/06

(57) ABSTRACT

Disclosed herein is a uterine collection device and method of using the same. The device has a generally tubular elongate shape for manipulation and material collection. A plunger and sheath extend from a proximal end to a distal end. At least one of the sheath and plunger includes three portions with progressively decreasing radii of curvature extending from the proximal end to the distal end. An optional syringe can be used. Rotating the uterine collection device to a point where the movement of the distal end of the uterine collection device is no longer frustrated by a variety of uterine positions is also disclosed.

### CROSS-REFERENCE TO RELAYED APPLICATIONS

This is the first application filed for the present invention.

### TECHNICAL FIELD

This application relates to uterine collection devices in general for taking cell samples of the uterus, including the cervix, and to an improved uterine collection device and method, in particular.

### BACKGROUND OF THE INVENTION

Methods or instruments for diagnosis by taking a cell sample for biopsy of the uterus, including the cervix, are known in the art.

For example, the Pipelle de Conner® manufactured by Prodimed of France, is well known in the art, and remains a reference. This instrument includes a flexible, transparent polypropylene sheath, approximately 3.10 mm in external diameter, 2.6 mm in internal diameter, and 23.5 cm long. A lateral orifice 2.1 mm in diameter is provided at its distal end, and four markings 4, 7, 8 and 10 cm from the distal end are typically provided. An internal plunger made of ethylyne-vinyl acetate (EVA) is provided which slides within the sheath and is stopped by the proximal end of the sheath. A flexible acetyl resin shaft is used to push the plunger. The entire instrument is usually sterilized with ethylene oxide inside a sterile packaging. It is know that the Pipelle de Cornier can be shaped before taking it out of its sterile packaging due to its slight elastic resistance that helps the device retain a convexity to fit a uterine anteflexion or retroflexion. Operationally, to take samples to screen for cancer, one would slide the device gently through the cervix to the uterine fundus. The marking 4 usually indicates the beginning of the uterine cavity, and unless there is an obstacle, the 7 mark can usually be reached. One would then draw back on the plunger to create negative pressure and sweep the uterine fundus multiple times while rotating the device to include the whole uterine cavity in the specimen. This process is stopped when fragments of uterine mucosa appear in the sheath, and the device is removed carefully after which the plunger is pushed to release the whole content in a recipient with a fixating solution.

As a further example, consider U.S. Pat. No. 6,491,645 entitled UTERINE TISSUE COLLECTOR, issued to Gaber on Dec. 10, 2002. Gaber cites a publication about the Pipelle de Cornier from 1984, and describes the sheath of the Pipelle de Cornier as having a “spaghetti-like limpness”, in the background of the invention section. Gaber discloses a uterine collector including a cannula which has an aperture formed therein through which tissue material can be sucked into a lumen formed into the cannula. At least one guide wire is attached to a distal portion of the cannula, wherein movement of the at least one guide wire flexes the distal portion of the cannula. A suction device operative to suck tissue material into the lumen is disclosed, comprising a syringe with a distal barrel which extends from a proximal barrel, the proximal barrel having a larger diameter than the distal barrel.

As a yet further example, consider U.S. patent application Ser. No. 11/851,227 entitled TUBAL CANNULATOR AND METHODS OF USE, published to Rotman et al. on Mar. 6, 2008. Rotman et al. cites Gaber in the background of invention, and states that the flexing or bending of the distal portion in Gaber does not appear to be permanent but instead is subject to continued force applied by the guide wires of Gaber. Rotman et al. disclose a cannulator that has an adjustable shape for conforming to anatomy to reach the tubal ostium of a patient. The cannulator includes a body segment, a tip section, and a handle. The body segment is made of a malleable material having a malleable characteristic with a degree of resistance to lateral deflection until sufficient force is applied to cause permanent bending thereby imparting a shat to the body segment. The tip section is positioned at a distal end of the body segment and the handle is positioned at a proximal end of the body segment. Gaber discloses at FIG. 1 that the cannulator including a tip section having a radius of curvature r. Confusingly, Rotman et al disclose that the tip section may have a radius, r, between 20 and 60 degrees and more particularly, approximately 30 degrees. An optional set of shaping mandrils is mentioned but not shown, and described as metal rods configured to fit within the lumen of the cannulator, however it is not clear how it is that a metal rod could change the radius of the tip of the cannulator only since it is inserted into the opposite end of the cannulator.

### SUMMARY

According to one aspect of the present application, there is provided a uterine collection device having a generally tubular elongate shape extending from a proximal end suitable for manipulation to a distal end suitable for material collection. The uterine collection device includes a plunger extending from the proximal end to the distal end; a sheath extending from the proximal end to the distal end having a transverse opening at the proximal end suitable for receiving the plunger, the distal end of the sheath forming a substantially closed transverse end, the closed transverse end of the sheath including a lateral orifice suitable for material collection through cause of displacement of the plunger within the sheath. At least one of the sheath and plunger includes at least three portions with progressively decreasing radii of curvature extending from the proximal end to the distal end. In some embodiments, the at least three portions include: a substantially flat portion provided at the proximal end having no appreciable curvature; a first curved portion provided between the proximal end and the distal end having a first radius of curvature R1 smaller than the radius of curvature of the substantially flat portion; and a second curved portion provided at the distal end having a second radius of curvature R2 smaller than the first radius of curvature R1. In alternative embodiments R1 is 200 mm and R2 is 200 mm. In some embodiments, at least three portions include: a substantially flat portion provided at the proximal end having a first length L0; a first curved portion provided between the proximal end and the distal end having a second length L1 larger than the first length L0; and a second curved portion provided at the distal end having a third length L2 smaller than the first length L0. In alternative embodiments, L0 is 70 mm, L1 is 135 mm, and L2 is 20 mm. In some embodiments, the sheath measures 3.10 mm in external diameter, 2.6 mm in internal diameter, 23.5 cm in length. In some embodiments, the lateral orifice measures 2.1 mm in diameter. In some embodiments, a handle is provided at the proximal end of the plunger for manipulating the plunger. In some embodiments, a stop provided at the proximal end of the sheath for stopping the displacement of the plunger. In some embodiments, the sheath further includes a second lateral orifice a the proximate end and the stop includes a port substantially aligned with the second lateral orifice suitable for receiving one of a syringe and a stopper. In alternative embodiments, a syringe is provided suitable for insertion into the port of the stop for providing additional pressure at the distal lateral orifice, or a stopper is provided suitable for insertion into the port of the stop. In some embodiments, the sheath is made of transparent propylene. In some embodiments, the plunger is made of ethelyne-vinyl acetate. In some embodiments, the handle is made of a flexible acetyl resin. In some embodiments, the sheath includes at least one marking indicative of a reference length suitable for at least one manipulation.

According to another aspect of the present application there is provided a method of use of a uterine collection device having a generally tubular elongate shape extending from a proximal end suitable for manipulation to a distal end suitable for material collection. The method includes the steps of: providing a plunger extending from the proximal end to the distal end; providing a sheath extending from the proximal end to the distal end having a transverse opening at the proximal end suitable for receiving the plunger, the distal end of the sheath forming a substantially closed transverse end, the closed transverse end of the sheath including a lateral orifice suitable for material collection through cause of displacement of the plunger within the sheath; and providing at least one of the sheath and plunger with at least three portions with progressively decreasing radii of curvature extending from the proximal end to the distal end. In some embodiments, the method comprises the step of providing at least one marking on the sheath indicative of a reference length suitable for at least one manipulation. In some embodiments, the method further comprises the steps of: inserting the uterine collection device into one of the vaginal canal and the uterine canal until such point that the movement of the distal end of the uterine collection device is frustrated; and rotating the uterine collection device to a point where the movement of the distal end of the uterine collection device is no longer frustrated. In some embodiments, the method further comprises the step of providing a port on the uterine collection device proximate the proximal end for receiving one of a stopper and a syringe. In some embodiments, the method comprises the step of using a syringe with the port for one of collecting material and flushing the uterine collection device.

Other aspects and features of the present invention will become apparent to those ordinarily skilled in the art upon review of the following description of specific embodiments of an improved uterine collection device and method in conjunction with the accompanying drawing figures.

### BRIEF DESCRIPTION OF THE DRAWINGS

Embodiments of the present invention will now be described, by way of example only, with reference to the accompanying drawing figures, wherein:

FIG. 1 is a perspective view of the components of a first embodiment of a uterine collection device;

FIG. 2A is a front view of the first embodiment of a uterine collection device of FIG. 1;

FIG. 2B is a side view of the first embodiment of a uterine collection device of FIG. 1;

FIG. 2C is a top view of the first embodiment of a uterine collection device of FIG. 1;

FIG. 3 is a perspective view of the first embodiment of a uterine collection device of FIG. 1;

FIG. 4A is a longitudinal sagital section of a woman showing the various axes of the uterine and vaginal canals, and pelvic brim and vaginal roof;

FIG. 4B is a longitudinal sagital section of a woman showing a normal position of female pelvic organs;

FIG. 4C is a longitudinal sagital section of a woman showing a diagrammatic of normal mobility of a uterus;

FIG. 4D is a longitudinal sagital section of a woman showing an uteral anteflexion, first degree;

FIG. 4E is a longitudinal sagital section of a woman showing an uteral anteflexion, second degree;

FIG. 4F is a longitudinal sagital section of a woman showing an uteral anteflexion, third degree;

FIG. 4G is a longitudinal sagital section of a woman showing an uteral anteflexion of the cervix;

FIG. 4H is a longitudinal sagital section of a woman showing an uteral anteflexion with retroposition;

FIG. 4I is a longitudinal sagital section of a woman showing an uteral retroflexion, second degree;

FIG. 4J is a longitudinal sagital section of a woman showing an uteral retroversion, first degree;

FIG. 4K is a longitudinal sagital section of a woman showing an uteral retroversion, second degree;

FIG. 4L is a longitudinal sagital section of a woman showing an uteral retroversion, third degree;

FIG. 5A is a front view of a plunger component of a second embodiment of a uterine collection device;

FIG. 5B is a side view of the plunger component of the second embodiment of a uterine collection device;

FIG. 5C is a top view of the plunger component of the second embodiment of a uterine collection device;

FIG. 6 is a perspective view of a plunger of the second embodiment of a uterine collection device;

FIG. 7A is a front view of a sheath of a second embodiment of a uterine collection device;

FIG. 7B is a side view of the sheath of the second embodiment of a uterine collection device;

FIG. 7C is a top view of the sheath of the second embodiment of a uterine collection device;

FIG. 8 is an exploded perspective view of the second embodiment of a uterine collection device;

FIG. 9 is an assembled perspective view of the second embodiment of a uterine collection device;

FIG. 10A is a front view of a sheath of a third embodiment of a uterine collection device;

FIG. 10B is a side view of the sheath of the third embodiment of a uterine collection device showing a compatible optional syringe;

FIG. 10C is a top view of the sheath of the third embodiment of a uterine collection device;

FIG. 11 is an exploded perspective view of the third embodiment of a uterine collection device showing the compatible optional syringe;

FIG. 12 is an assembled perspective view of the third embodiment of a uterine collection device showing the compatible optional syringe at a disconnected position;

FIG. 13 is an assembled perspective view of the third embodiment of a uterine collection device showing the compatible optional syringe at a connected position;

FIG. 14A is a front view of a sheath of a fourth embodiment of a uterine collection device;

FIG. 14B is a side view of the sheath of the fourth embodiment of a uterine collection device showing a compatible optional syringe;

FIG. 14C is a top view of the sheath of the fourth embodiment of a uterine collection device;

FIG. 15 is an exploded perspective view of the fourth embodiment of a uterine collection device showing the compatible optional syringe;

FIG. 16 is an assembled perspective view of the fourth embodiment of a uterine collection device showing the compatible optional syringe at a disconnected position;

FIG. 17 is an assembled perspective view of the fourth embodiment of a uterine collection device showing the compatible optional syringe at a connected position;

FIG. 18A is a perspective view of the components of a fifth embodiment of a uterine collection device showing a compatible optional syringe;

FIG. 18B is an enlarged view of a portion of FIG. 18A;

FIG. 19A is a front view of the fifth embodiment of a uterine collection device of FIG. 18A;

FIG. 19B is a side view of the fifth embodiment of a uterine collection device of FIG. 18A;

FIG. 19C is a top view of the fifth embodiment of a uterine collection device of FIG. 18A;

FIG. 20 is an assembled perspective view of the fifth embodiment of a uterine collection device showing the compatible optional syringe at a disconnected position; and

FIG. 21 illustrates an exemplary embodiment of a method of use of a uterine collection device.

Like reference numerals are used in different figures to denote similar elements.

### DETAILED DESCRIPTION OF THE DRAWINGS

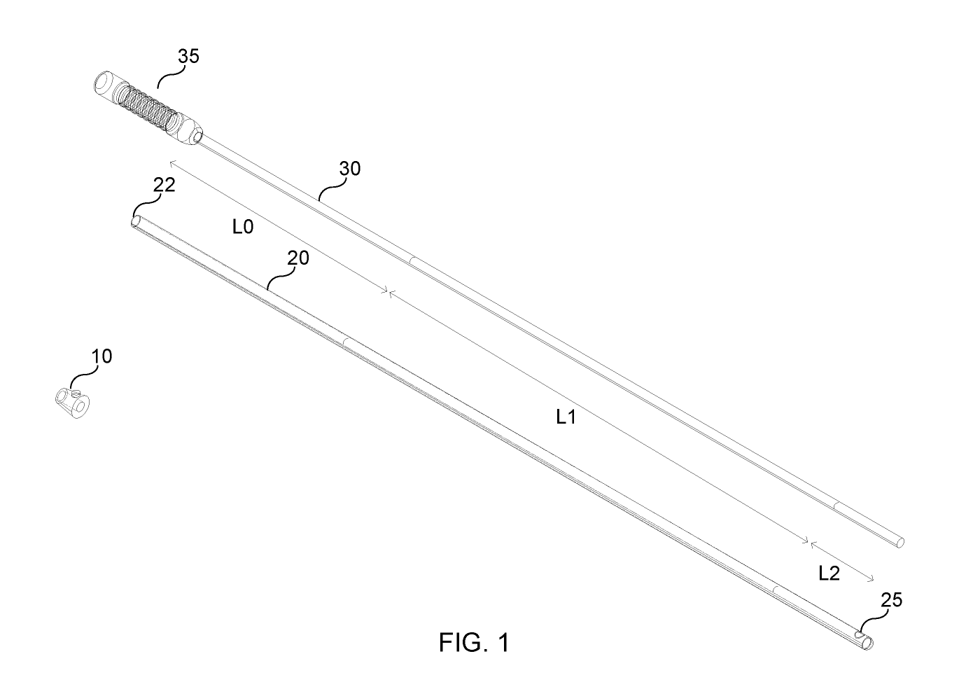

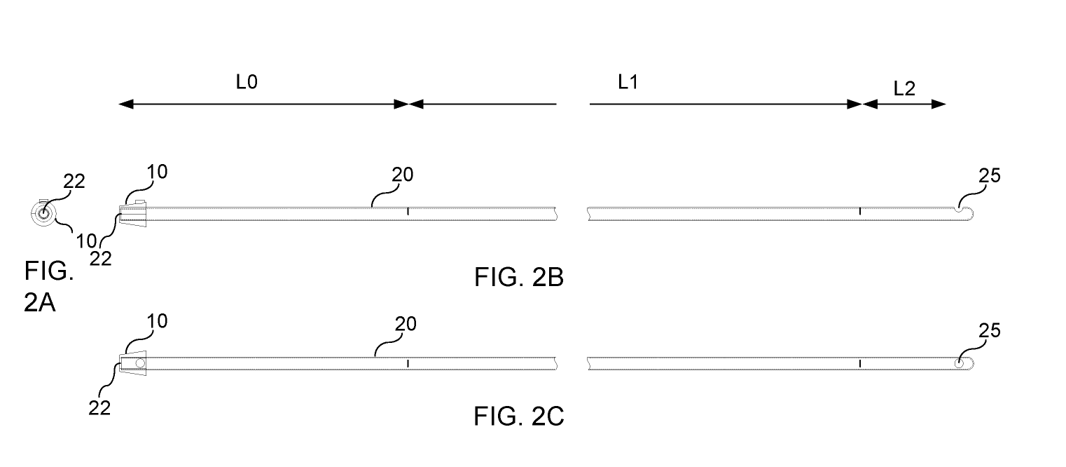

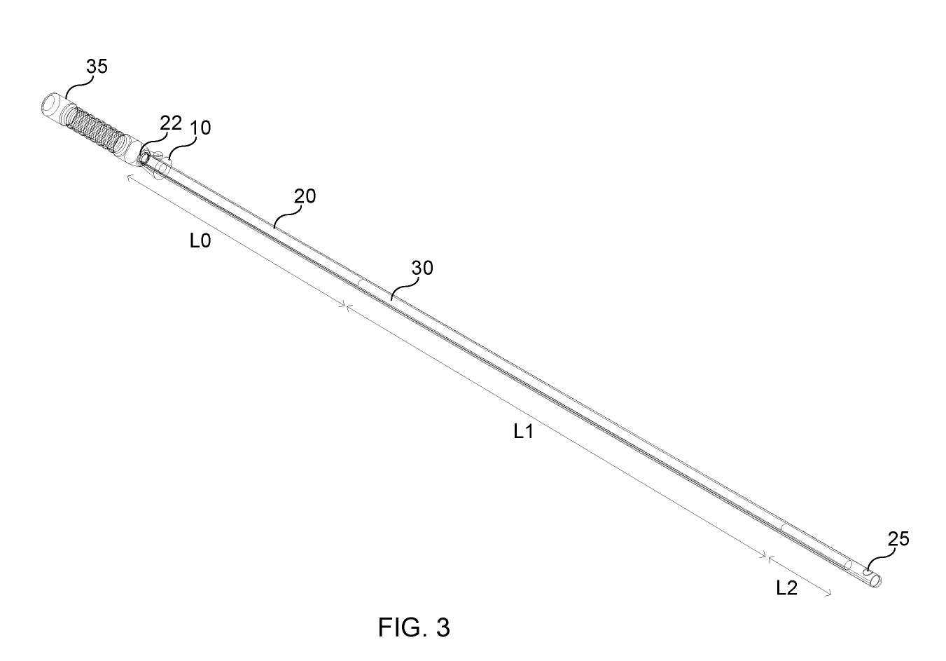

Referring to the drawings, FIG. 1 is a perspective view of the components of a first embodiment of a uterine collection device. As shown, the main components include a stop 10, a sheath 20, and a plunger 30. The sheath 20 is provided with a transverse opening 22 near the proximal or open end, and a lateral orifice 25 near the distal or closed end. The sheath 20 is preferably made of a flexible material such as for example transparent polypropylene, and in a preferred embodiment measures approximately 3.10 mm in external diameter, 2.6 mm in internal diameter, and 23.5 cm long. The lateral orifice 25 preferably measures 2.1 mm in diameter. The transverse opening 22 preferably measures 2.6 mm in diameter. Optionally, markings at fixed reference lengths such as L2, L1 and L0 from the distal end of the plunger 30 can be provided on the sheath 20 as reference points for various manipulations. The stop 10 can be of any shape so long as it can be affixed to the proximal end of the sheath 20 so as to limit the distance that the plunger 30 can enter the sheath, and as illustrated includes a relief, substantially similar in diameter to the lateral orifice 25, and is preferably placed such that the relief is oriented in the same direction as the lateral orifice 25 in order to facilitate manipulating the sheath 20 to orient the lateral orifice 25. The plunger 30 is provided with a handle 35 at the proximal end which is stopped by the stop 10. The plunger 30 is preferably made of a flexible material, such as for example, ethylyne-vinyl acetate (EVA), and slides within the sheath 20 and is stopped by the proximal end of the sheath 20 by the stop 10 frustrating the motion of the handle 35 and to provide a way of conveying force to the sheath when withdrawing the plunger by pulling on the handle 35. At the position where the plunger 30 is fully inserted into the sheath 20, the length of the plunger is smaller than that of the sheath such that there is a portion of the sheath near the distal end that acts as a lumen under lateral orifice 25. The handle 35 of the plunger 30 can be made of a suitable material, such as for example acetyl resin to be able to push the plunger 30. The components can be sterilized with ethylene oxide. FIG. 2A is a front view of the first embodiment of a uterine collection device of FIG. 1. FIG. 2B is a side view of the first embodiment of a uterine collection device of FIG. 1. FIG. 2C is a top view of the first embodiment of a uterine collection device of FIG. 1. FIG. 3 is a perspective view of the first embodiment of a uterine collection device of FIG. 1. The components of the first embodiment of a uterine collection device of FIGS. 1-3 could be utilised, for example, to provide a device that is functionally equivalent to the Pipelle de Cornier.

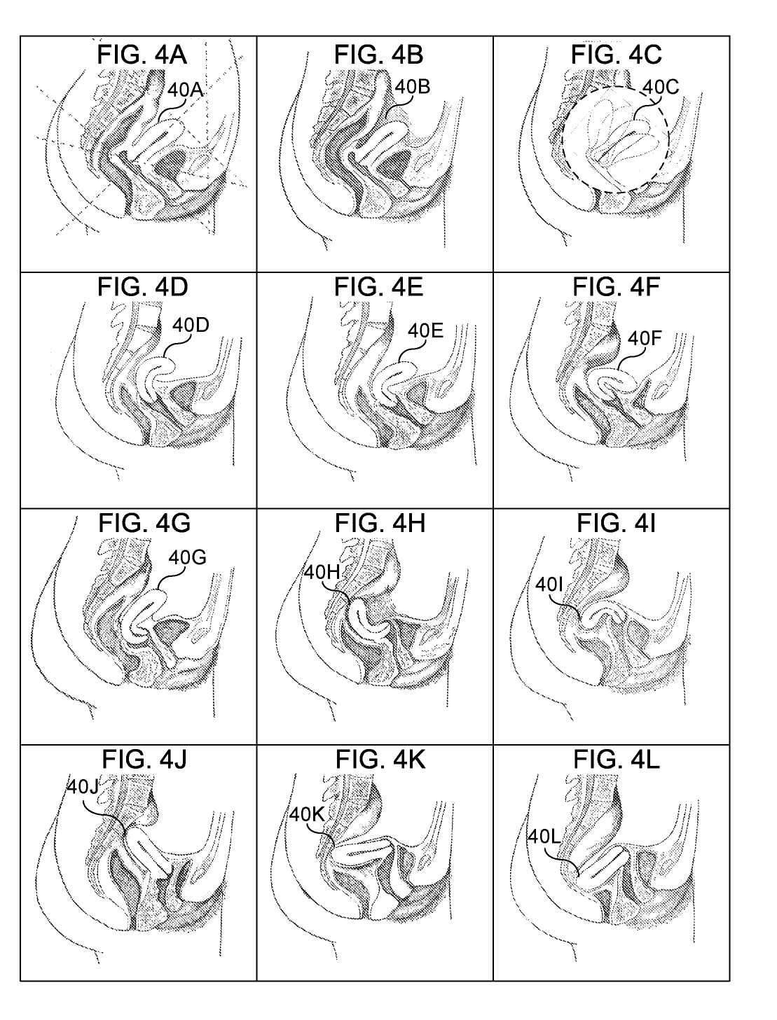

However, the embodiment of a uterine collection device of FIGS. 1-3 may not be able to be used, without further improvement, in order to sample all of the different anatomical variations of uterine positions that a physician may encounter, such as those shown in FIGS. 4A-4L. FIG. 4A is a longitudinal sagital section of a woman showing the various axes of the uterine and vaginal canals, and pelvic brim and vaginal roof. As illustrated the vaginal canal is at a substantially right angle with uterine canal, as shown by the doted lines that intersect where the vaginal canal meets the uterine canal such that access to the uterus 40A may be difficult with the first embodiment of the uterine device of FIGS. 1-3, without further improvement. FIG. 4B is a longitudinal sagital section of a woman showing a normal position of female pelvic organs. Access to the normal uterus 40B may be difficult with the first embodiment of the uterine device of FIGS. 1-3, without further improvement. FIG. 4C is a longitudinal sagital section of a woman showing a diagrammatic of normal mobility of a uterus. Access to the normally mobile uterus 40C may be difficult with the first embodiment of the uterine device of FIGS. 1-3, without further improvement. FIG. 4D is a longitudinal sagital section of a woman showing an uteral anteflexion, first degree. Access to the first degree anteflexed uterus 40D may be difficult with the first embodiment of the uterine device of FIGS. 1-3, without further improvement. FIG. 4E is a longitudinal sagital section of a woman showing an uteral anteflexion, second degree. Access to the second degree anteflexed uterus 40E may be difficult with the first embodiment of the uterine device of FIGS. 1-3, without further improvement. FIG. 4F is a longitudinal sagital section of a woman showing an uteral anteflexion, third degree. Access to the third degree anteflexed uterus 40F may be difficult with the first embodiment of the uterine device of FIGS. 1-3, without further improvement. FIG. 4G is a longitudinal sagital section of a woman showing an uteral anteflexion of the cervix. Access to the uterus having an anteflexion of the cervix 40G may be difficult with the first embodiment of the uterine device of FIGS. 1-3, without further improvement. FIG. 4H is a longitudinal sagital section of a woman showing an uteral anteflexion with retroposition. Access to the uterus having anteflexion with retroposition 40H may be difficult with the first embodiment of the uterine device of FIGS. 1-3, without further improvement. FIG. 4I is a longitudinal sagital section of a woman showing an uteral retroflexion, second degree. Access to the second degree retroflexed uterus 40I may be difficult with the first embodiment of the uterine device of FIGS. 1-3, without further improvement. FIG. 4J is a longitudinal sagital section of a woman showing an uteral retroversion, first degree. Access to the uterus first degree retroverted uterus 40J may be difficult with the first embodiment of the uterine device of FIGS. 1-3, without further improvement. FIG. 4K is a longitudinal sagital section of a woman showing an uteral retroversion, second degree. Access to the second degree retroverted uterus 40K may be difficult with the first embodiment of the uterine device of FIGS. 1-3, without further improvement FIG. 4L is a longitudinal sagital section of a woman showing an uteral retroversion, third degree. Access to the third degree retroverted uterus 40L may be difficult with the first embodiment of the uterine device of FIGS. 1-3, without further improvement.

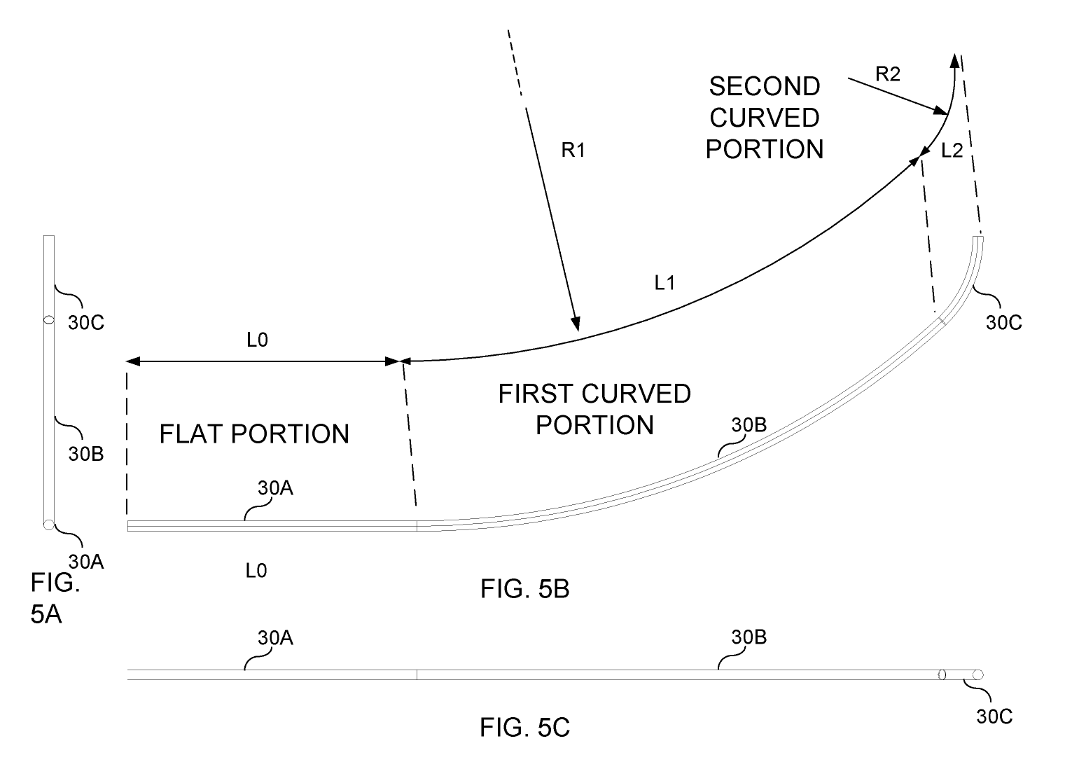

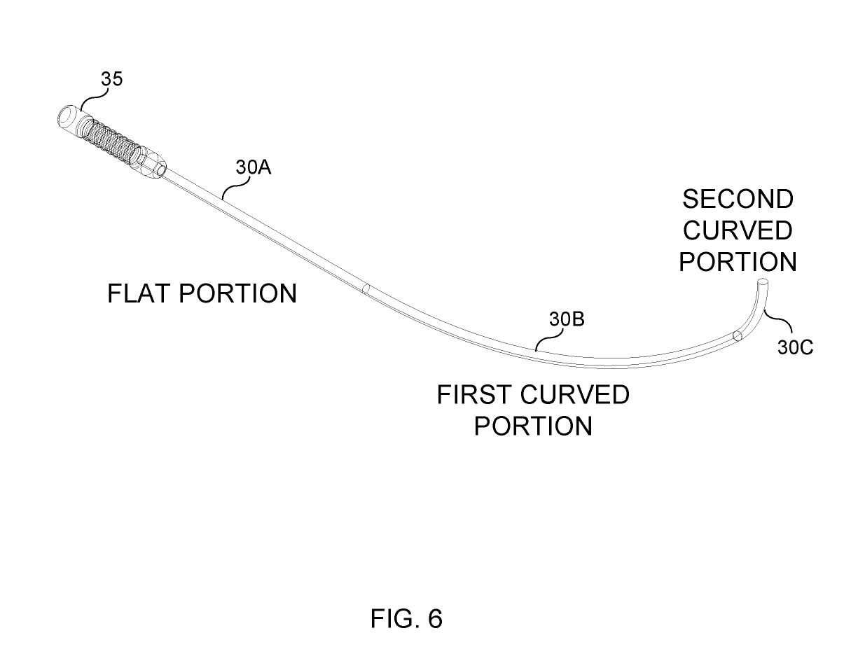

FIG. 5A is a front view of a plunger component of a second embodiment of a uterine collection device. FIG. 5B is a side view of the plunger component of the second embodiment of a uterine collection device. FIG. 5C is a top view of the plunger component of the second embodiment of a uterine collection device. FIG. 6 is a perspective view of a plunger of the second embodiment of a uterine collection device. The plunger component includes a flat portion 30A, a first curved portion 30B, and a second curved portion 30C. The first curved portion has a first radius of curvature R1. The second curved portion has a second radius of curvature R2. As illustrated the length of the first curved portion L1 is greater than the length of the flat portion L0 which is in turn greater than the length of the second curved portion L2. The first radius of curvature R1 is greater than the second radius of curvature R2. Although not expressly shown in the drawing, in an alternative embodiment, the flat portion need not be completely flat and may have a radius of curvature R0 which is very large relative to R1 and R2. Advantageously, the three portions of the plunger component have progressively increasing radii of curvature R0, R1 and R2 and are dimensioned with lengths L0, L1 and L2 so as to provide improved access to the various uteri of FIGS. 4A-4L. For example, in FIGS. 5A-5C and 6, L0 is 70 mm and R0 is infinite, L1 is 135 mm and R1 is 200 mm, and L2 is 20 mm and R2 is 20 mm. These values are exemplary only and are a preferred embodiment. It is envisaged that a person of ordinary skill in the art may vary these values to suit their preference without undue experimentation, and any such variation is understood to be within the scope of the present application. A handle 35 is provided at the proximal end of the plunger 30.

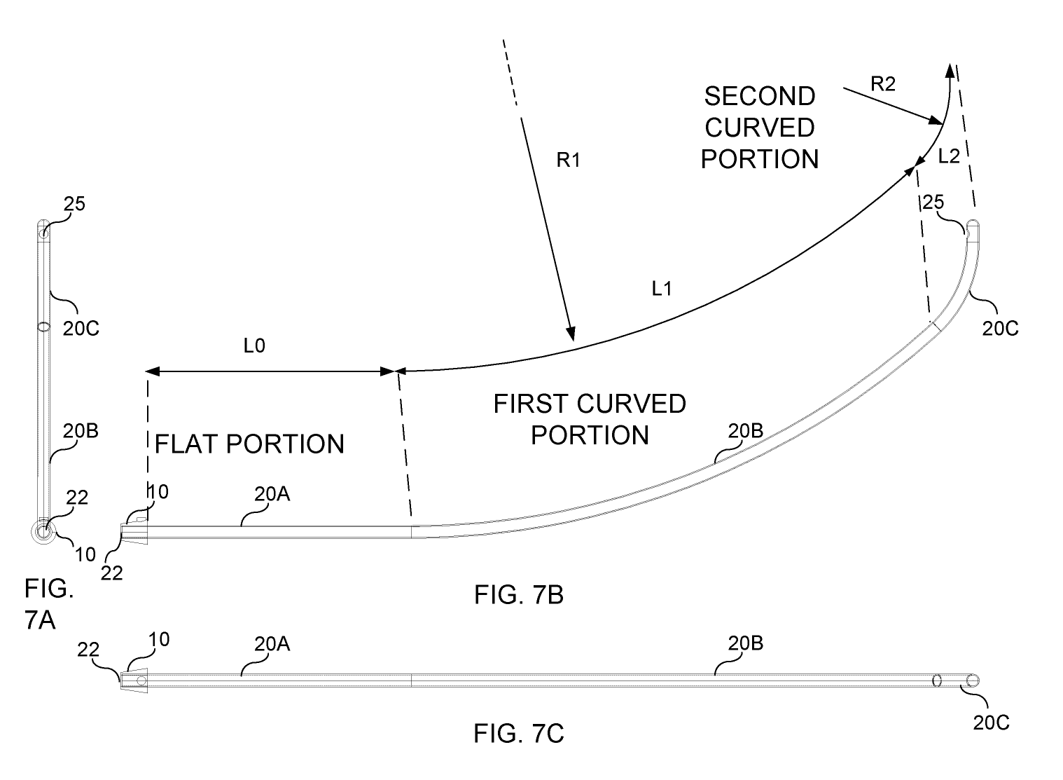

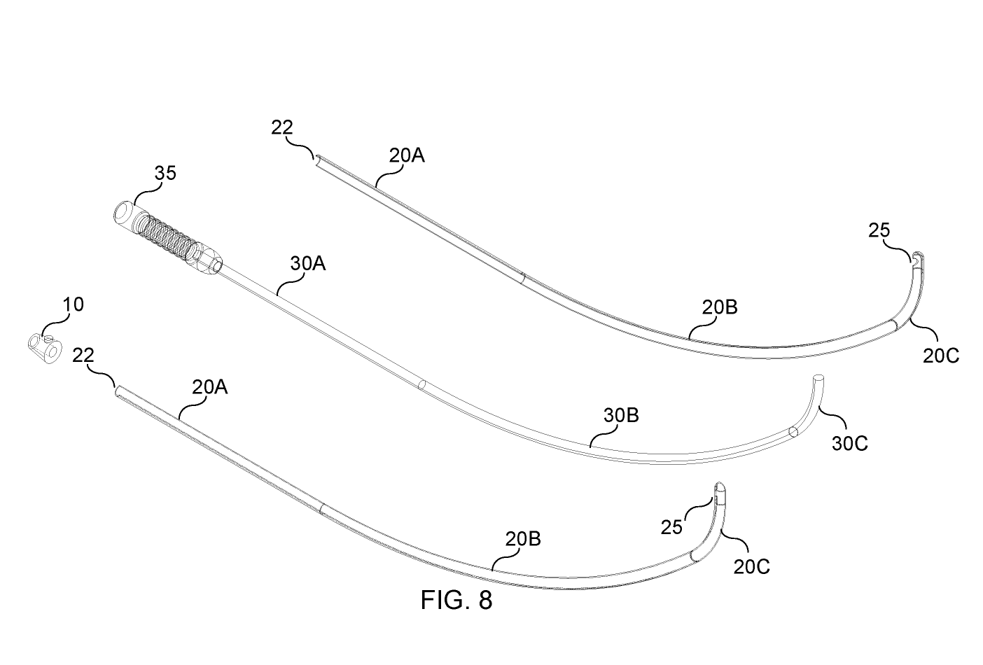

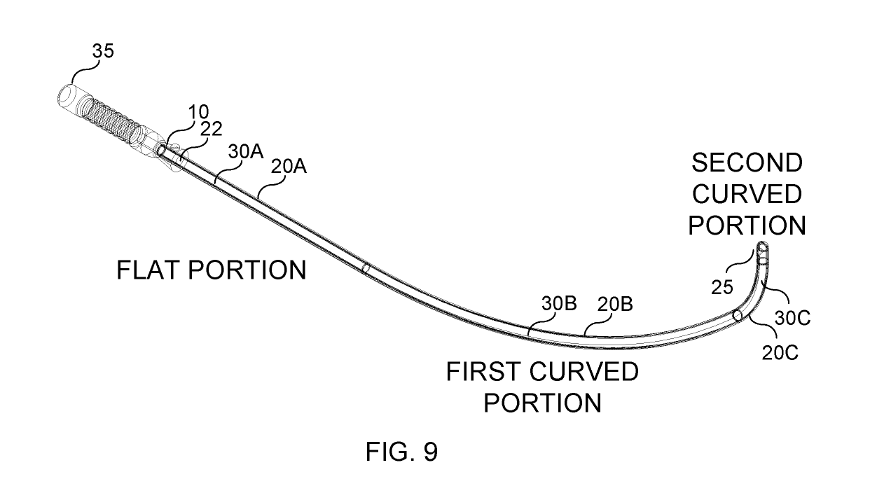

FIG. 7A is a front view of a sheath of a second embodiment of a uterine collection device. FIG. 7B is a side view of the sheath of the second embodiment of a uterine collection device. FIG. 7C is a top view of the sheath of the second embodiment of a uterine collection device. FIG. 8 is an exploded perspective view of the second embodiment of a uterine collection device. FIG. 9 is an assembled perspective view of the second embodiment of a uterine collection device. As with the plunger component of FIGS. 5A-5C and 6, the sheath includes a flat portion 20A, a first curved portion 20B, and a second curved portion 20C.

In the illustrated examples of FIGS. 1-9, the sheath 20, the handle 35 and the stop 10 of FIGS. 1-3, can be used to provide the flat portions 20A and 30A, first curved portions 20A and 30B, and second curved portions 20C and 30C of the sheath and plunger respectively of FIGS. 5-9. Advantageously, the two markings at positions L0 and L0+L1 from the proximal end can be used to shape the plunger component 30 and sheath 20 of FIGS. 1-3 to provide the flat portions 20A and 30A, first curved portions 20B and 30B, and second curved portions 20C and 30C of the plunger and sheath of FIGS. 5-9. Operationally, in order to shape the plunger and sheath at the first curved portion, one holds the plunger and sheath at or near a fixed distance L0 from the proximal end of the plunger handle with the thumb and index of one hand, and applies an upwards pressure while sliding the thumb and index of the other hand from the fixed distance L0 towards the distal end of the plunger and sheath until the desired radius of curvature R1 of the first curved portions is reached. The process is then repeated with the thumb and index of one hand holding the plunger and sheath at or near a second fixed distance L0+L1 from the proximal end of the plunger handle, and applying an upwards pressure while sliding the thumb and index of the other hand from that fixed distance towards the distal end of the plunger and sheath. Advantageously, the markings provide reference points for effecting these manipulations, which can be accomplished while the plunger and sheath are still in their packaging.

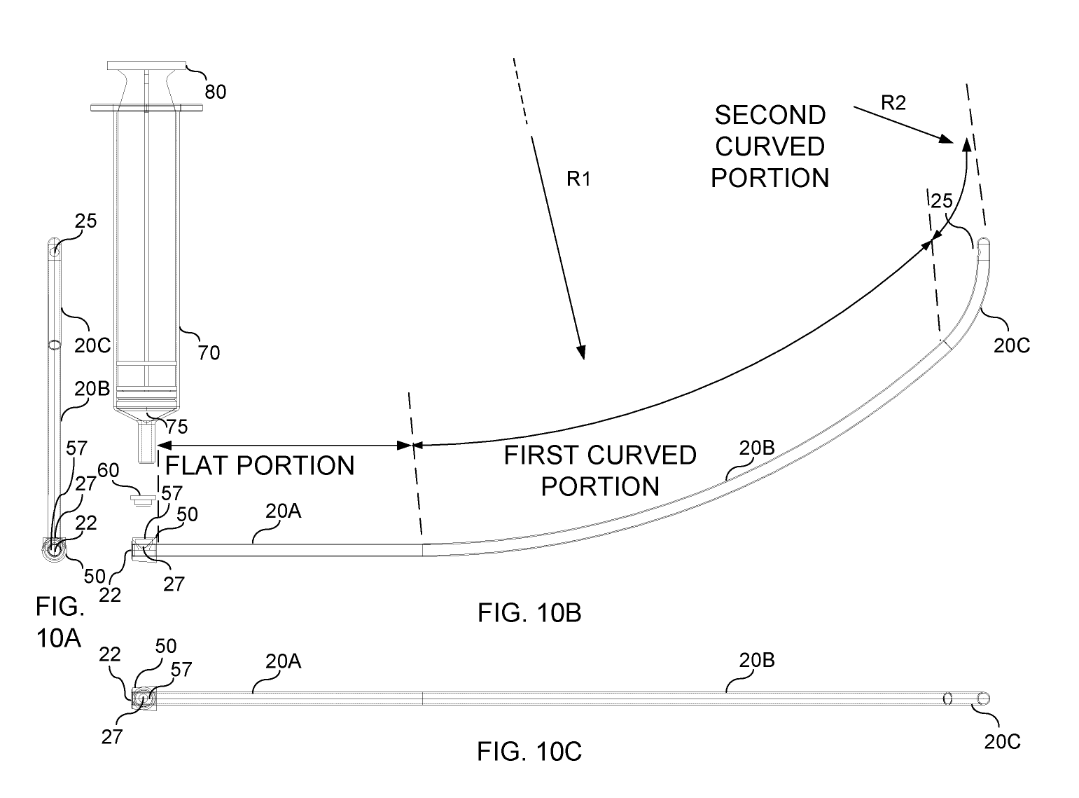

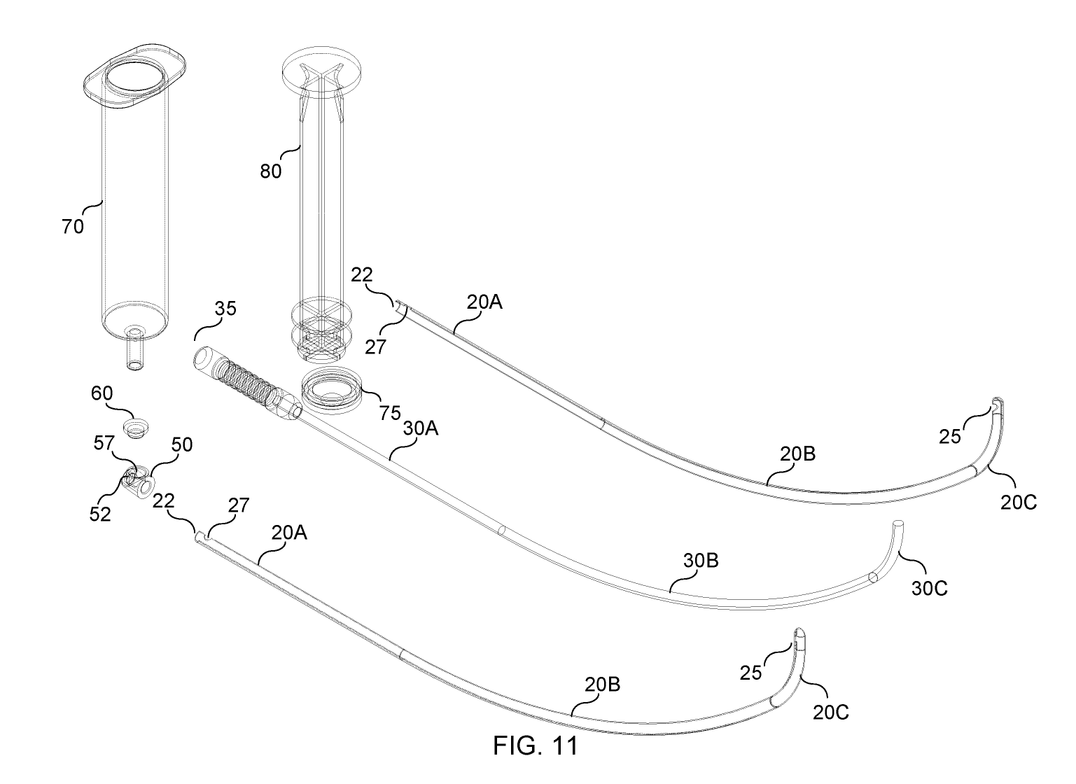

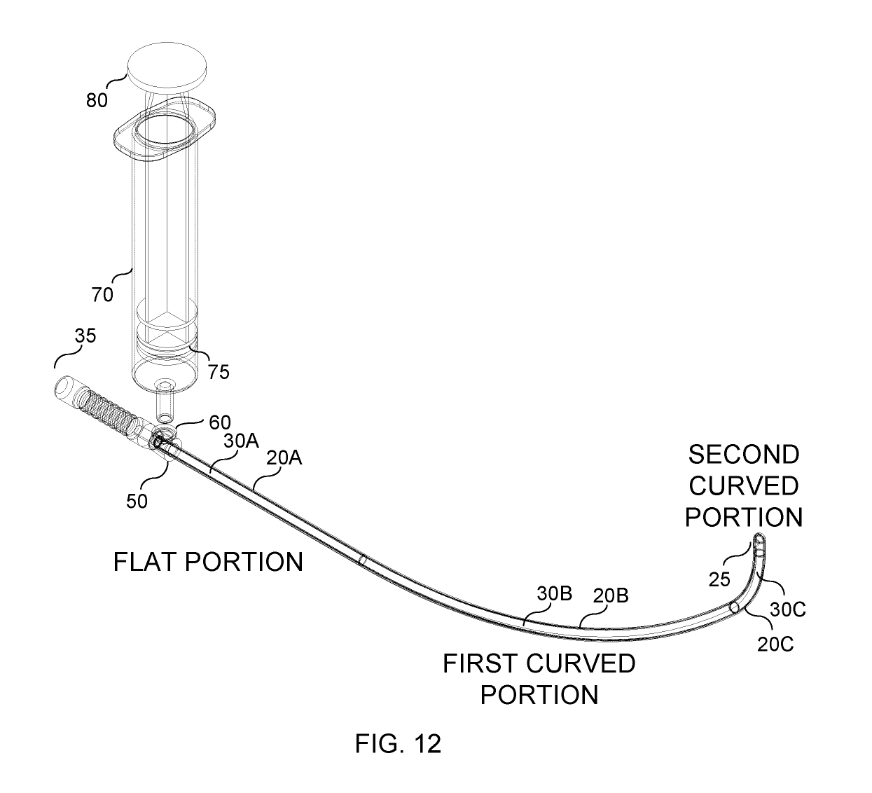

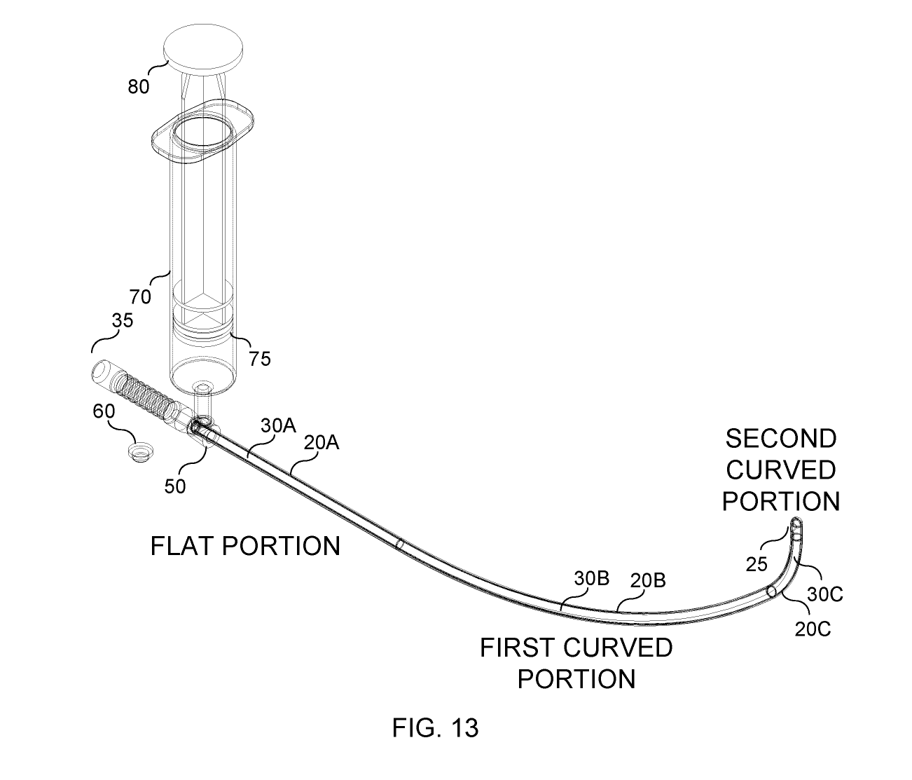

FIG. 10A is a front view of a sheath of a third embodiment of a uterine collection device. FIG. 10B is a side view of the sheath of the third embodiment of a uterine collection device showing a compatible optional syringe. FIG. 10C is a top view of the sheath of the third embodiment of a uterine collection device. As with the second embodiment of FIGS. 7A-7C, FIGS. 8-9, the sheath includes a flat portion 20A, a first curved portion 20B, and a second curved portion 20C. However, instead of including a stop 10, a port stop 50 is provided for use with an optional syringe. To that end, a proximal lateral orifice 27 is provided at the proximal end of the flat portion 20A of the sheath, substantially similar in size as the distal lateral orifice 25, coincidental with port 57 of port stop 50. The syringe includes syringe cylinder 70, syringe plunger 80, and syringe gasket 75. When used, advantageously the syringe connects the lumen of the sheath 20 to the lumen inside the cylinder 70 of the syringe via the proximal lateral orifice 27 thereby providing additional suction at the distal lateral orifice 25. Furthermore, when the sheath is filled with samples, operation of the syringe can be used to evacuate the material into the syringe lumen by pulling on the plunger 80, or to evacuate the material out of the sheath via distal orifice 27 by pushing on the plunger 80. When the syringe is not in use, advantageously, port 57 of port stop 50 can be blocked using stopper 60. FIG. 11 is an exploded perspective view of the third embodiment of a uterine collection device showing the compatible optional syringe. FIG. 12 is an assembled perspective view of the third embodiment of a uterine collection device showing the compatible optional syringe at a disconnected position. FIG. 13 is an assembled perspective view of the third embodiment of a uterine collection device showing the compatible optional syringe at a connected position.

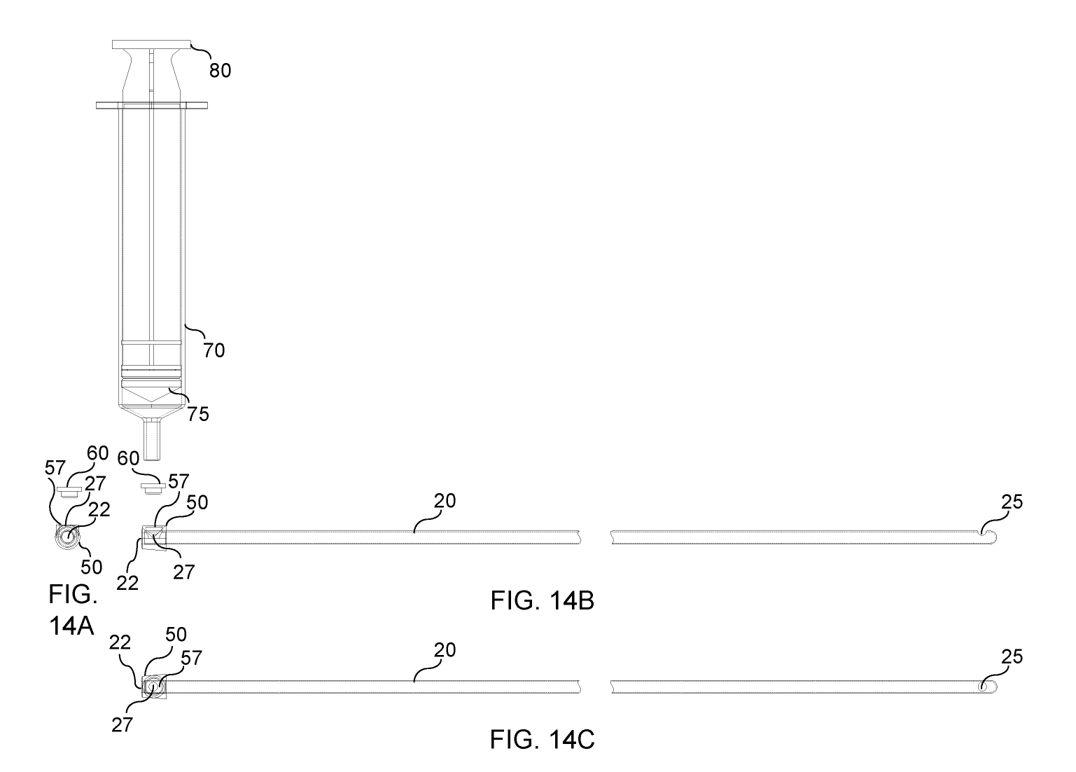

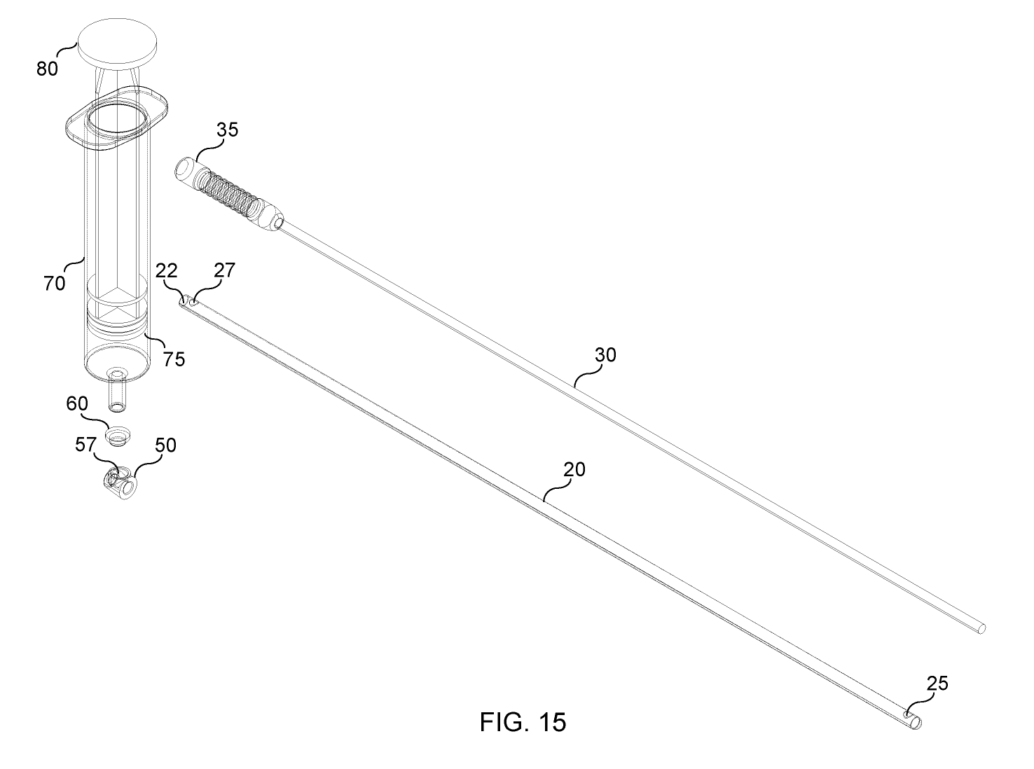

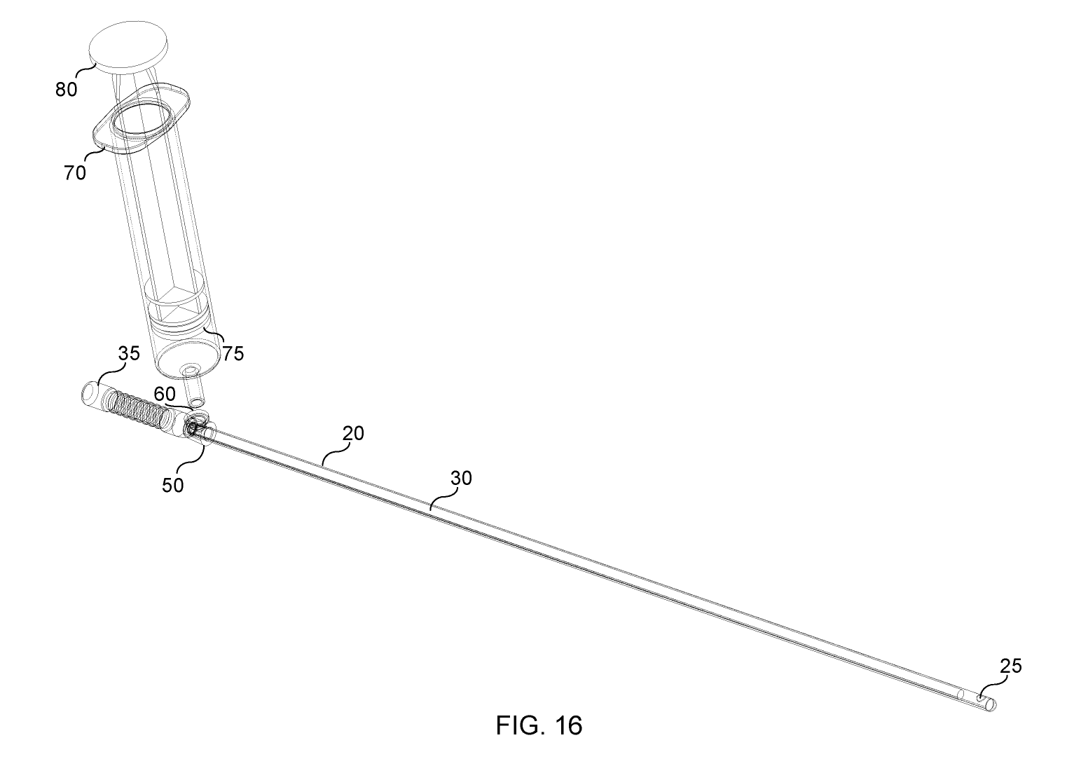

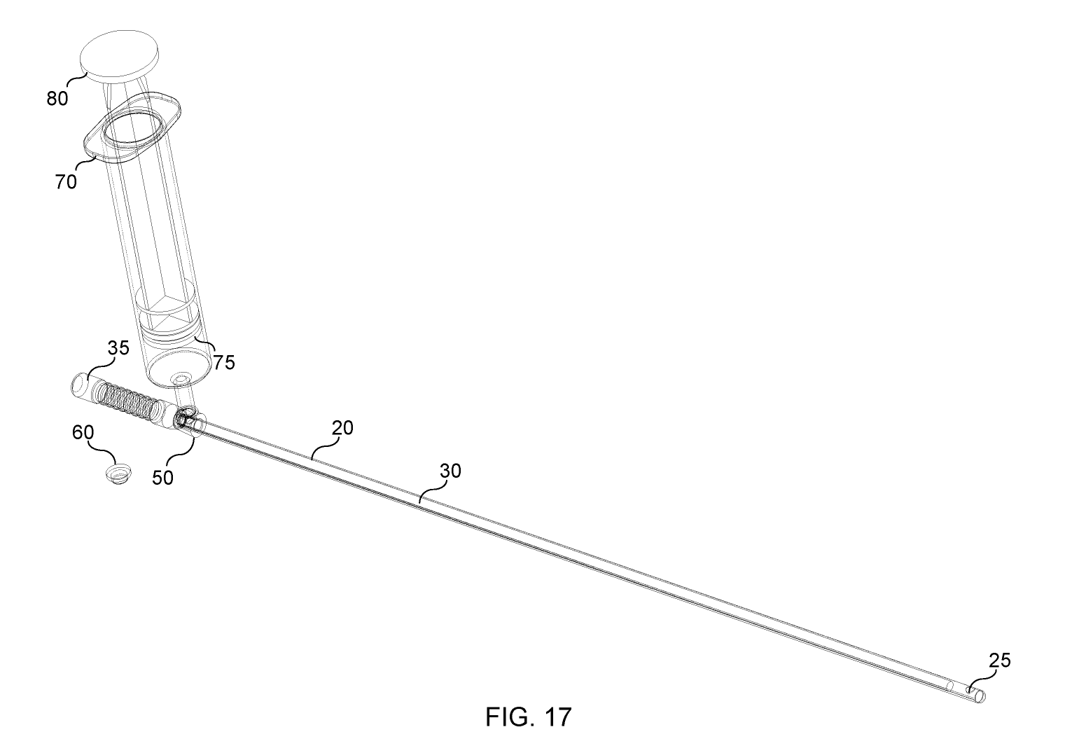

FIG. 14A is a front view of a sheath of a fourth embodiment of a uterine collection device. FIG. 14B is a side view of the sheath of the fourth embodiment of a uterine collection device showing a compatible optional syringe. FIG. 14C is a top view of the sheath of the fourth embodiment of a uterine collection device. FIG. 15 is an exploded perspective view of the fourth embodiment of a uterine collection device showing the compatible optional syringe. FIG. 16 is an assembled perspective view of the fourth embodiment of a uterine collection device showing the compatible optional syringe at a disconnected position. FIG. 17 is an assembled perspective view of the fourth embodiment of a uterine collection device showing the compatible optional syringe at a connected position. This fourth embodiment applies the port stop 50 and optional syringe cylinder 70, syringe plunger 80, and syringe gasket 75 features to the first embodiment.

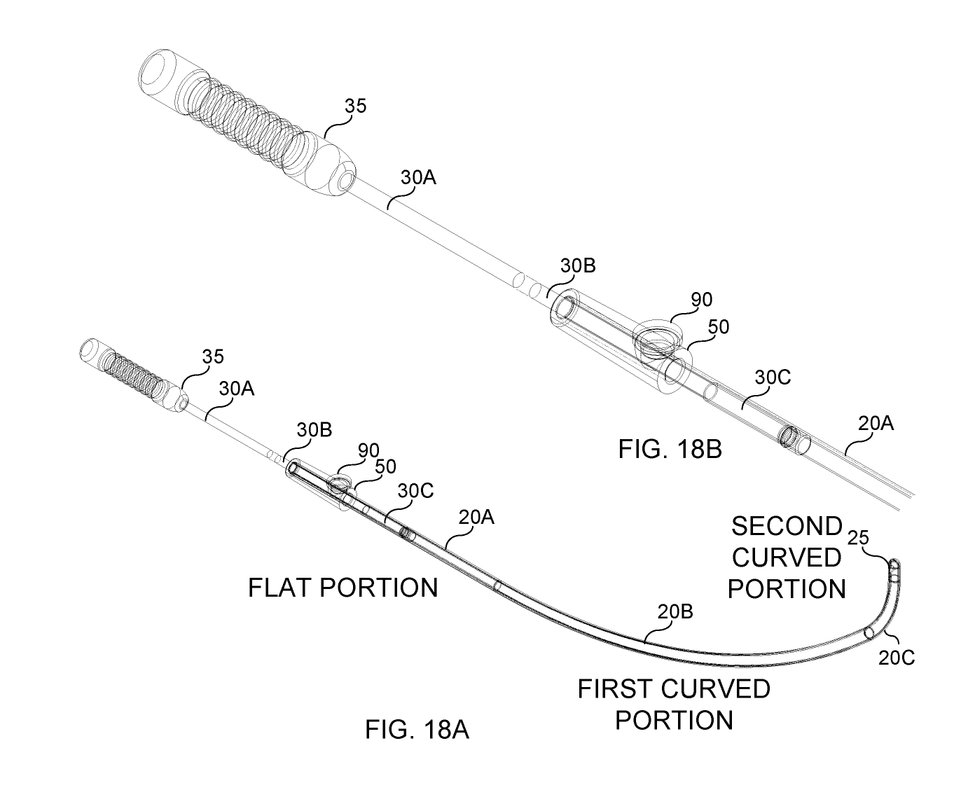

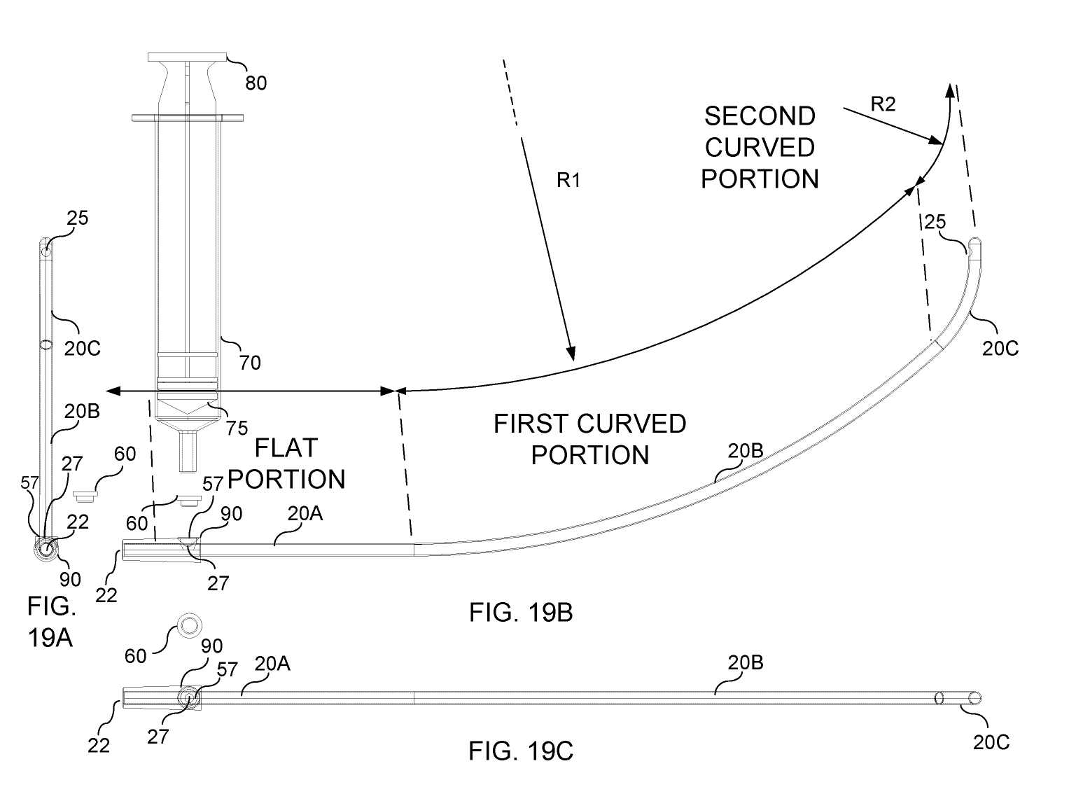

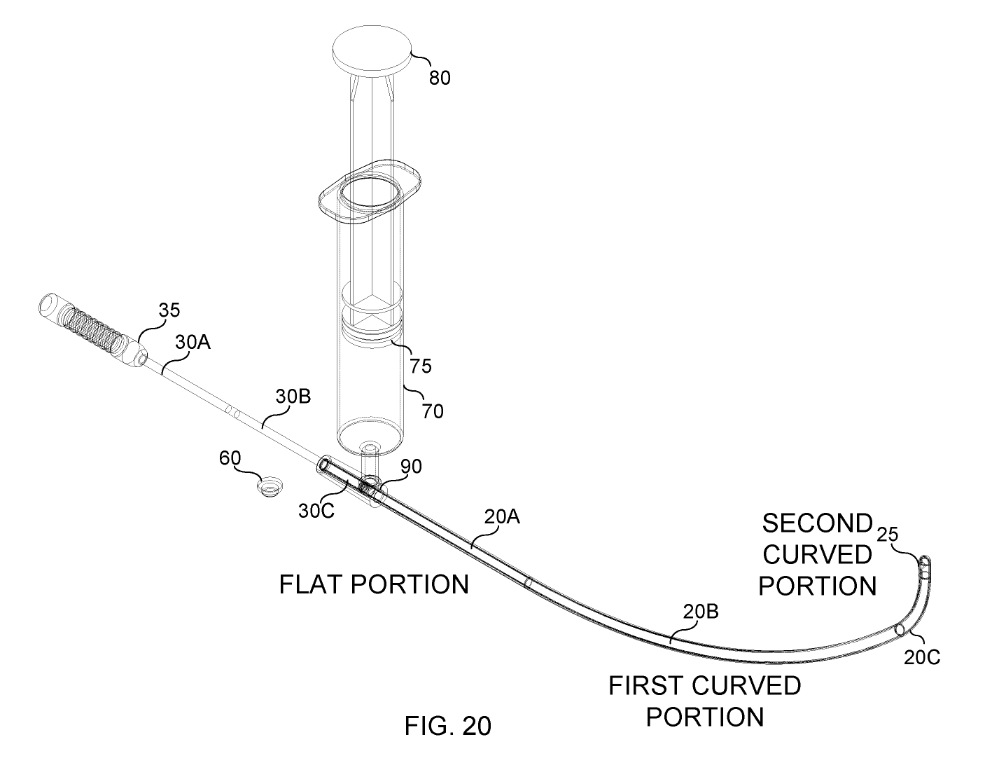

FIG. 18A is a perspective view of the components of a fifth embodiment of a uterine collection device showing a compatible optional syringe. FIG. 18B is an enlarged view of a portion of FIG. 18A. FIG. 19A is a front view of the fifth embodiment of a uterine collection device of FIG. 18A. FIG. 19B is a side view of the fifth embodiment of a uterine collection device of FIG. 18A. FIG. 19C is a top view of the fifth embodiment of a uterine collection device of FIG. 18A. FIG. 20 is an assembled perspective view of the fifth embodiment of a uterine collection device showing the compatible optional syringe at a disconnected position. In this fifth embodiment, an elongated port stop 90 is provided instead of a regular port stop 50. An advantage our the elongated port stop 90 is that the second curved portion 30C of the plunger can be retracted past the proximal lateral orifice 27 and port 57, thereby providing a more direct access to communicating the lumen in the cylinder 70 of the syringe with the lumen of the sheath 20.

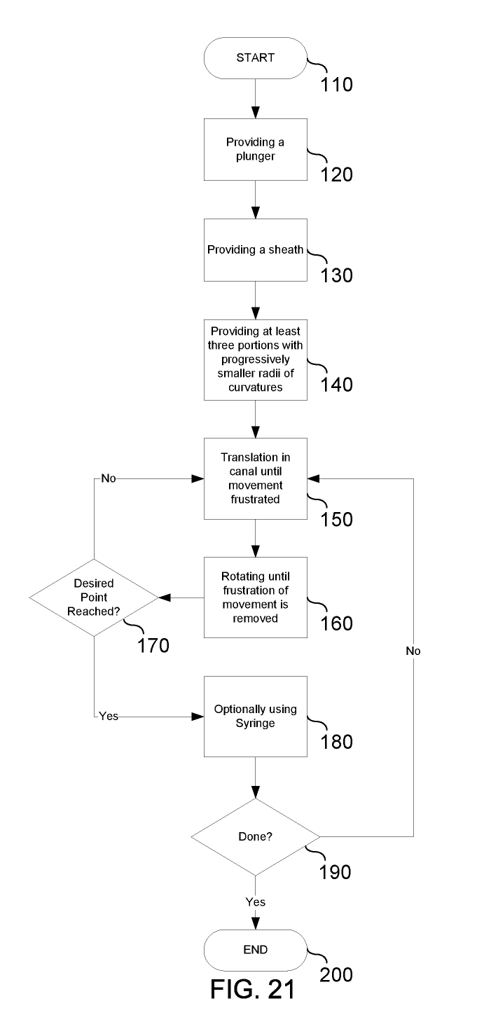

FIG. 21 illustrates an exemplary embodiment of a method of use of a uterine collection device. At step 110, the method starts. Step 120 includes the act of providing a plunger in the uterine collection device. Step 130 includes the act of providing a sheath in the uterine collection device. Step 140 includes the act of providing at least three portions with progressively smaller radii of curvatures of the uterine collection device. Step 150 includes the act of translation in a canal until movement is frustrated of the uterine collection device, such as for example, due to any of the uterine positions illustrated in FIGS. 4A-4L, either when moving inwardly while inserting the uterine collection device, or outwardly when removing the uterine collection device. Step 160 includes the act of rotating until frustration of movement is removed for the uterine collection device. For example, this may include a rotation of 90 degrees along the transverse axis of the handle so as to overcome any uterine positions illustrated in FIGS. 4A-4L. Other angles of rotation are envisaged if, for example uterine positions present in a manner that is not as illustrated in the examples. Step 170 includes the determination if a desired point has been reached such as for example a uterine collection point for material collection or removal of the device once material has been collected. If so determined, step 180 ensues, alternatively step 150 ensues. Step 180 includes the optional act of using a syringe with the uterine collection device if a port is provided on the stop of the uterine collection device. For example this could be at the collection point to increase suction and collection of material, or when the uterine collection device has been removed to flush the uterine collection device. Step 190 includes the act of determining if manipulations are done. For example, at this point the uterine collection device may be at a position for collection, in which case the next step 150 would ensue to remove the uterine collection device. Alternatively, if all manipulations are complete, step 100 ensues representing the end of the method.

Operationally, the uterine collection device has a generally tubular elongate shape extending from a proximal end suitable for manipulation to a distal end suitable for material collection. The steps of operation include providing a plunger extending from the proximal end to the distal end, providing a sheath extending from the proximal end to the distal end having a transverse opening at the proximal end suitable for receiving the plunger, the distal end of the sheath forming a substantially closed transverse end, the closed transverse end of the sheath including a lateral orifice suitable for material collection through cause of displacement of the plunger within the sheath; and providing at least one of the sheath and plunger with at least three portions with progressively decreasing radii of curvature extending from the proximal end to the distal end. In some embodiments, at least one marking is provided on the sheath indicative of a reference length suitable for at least one manipulation. In some embodiments, the method includes inserting the uterine collection device into one of the vaginal canal and the uterine canal until such point that the movement of the distal end of the uterine collection device is frustrated; and rotating the uterine collection device to a point where the movement of the distal end of the uterine collection device is no longer frustrated. In some embodiments a port is provided and a syringe is optionally used to aid in material collection either when the uterine collection device is at a desired position inside the uterine canal or outside the body to flush the uterine device.

The above-described embodiments of the present invention are intended to be examples only. Those of skill in the art may effect alterations, modifications and variations to the particular embodiments without departing from the scope of the invention, which is set forth in the claims.

What is claimed is:

1. A uterine collection device having a generally tubular elongate shape extending from a proximal end suitable for manipulation to a distal end suitable for material collection, the uterine collection device comprising:
a plunger extending from the proximal end to the distal end;
a sheath extending from the proximal end to the distal end having a transverse opening at the proximal end suitable for receiving the plunger, the distal end of the sheath forming a substantially closed transverse end, the closed transverse end of the sheath including a lateral orifice suitable for material collection through cause of displacement of the plunger within the sheath;
wherein at least one of the sheath and plunger comprises at least three portions with progressively decreasing radii of curvature extending from the proximal end to the distal end.

2. The uterine collection device of claim 1, wherein the at least three portions include:
a substantially flat portion provided at the proximal end having no appreciable curvature;
a first curved portion provided between the proximal end and the distal end having a first radius of curvature R1 smaller than the radius of curvature of the substantially flat portion; and
a second curved portion provided at the distal end having a second radius of curvature R2 smaller than the first radius of curvature R1.

3. The uterine collection device of claim 2, wherein R1 is 200 mm.

4. The uterine collection device of claim 2, wherein R2 is 200 mm.

5. The uterine collection device of claim 1, wherein the at least three portions include:
a substantially flat portion provided at the proximal end having a first length L0;
a first curved portion provided between the proximal end and the distal end having a second length L1 larger than the first length L0; and
a second curved portion provided at the distal end having a third length L2 smaller than the first length L0.

6. The uterine collection device of claim 5, wherein L0 is 70 mm.

7. The uterine collection device of claim 5, wherein L1 is 135 mm.

8. The uterine collection device of claim 5, wherein L2 is 20 mm.

9. The uterine collection device of claim 1, wherein the sheath measures 3.10 mm in external diameter.

10. The uterine collection device of claim 1, wherein the sheath measures 2.6 mm in internal diameter.

11. The uterine collection device of claim 1, wherein the sheath measures 23.5 cm in length.

12. The uterine collection device of claim 1, wherein the lateral orifice measures 2.1 mm in diameter.

13. The uterine collection device of claim 1, further comprising a handle provided at the proximal end of the plunger for manipulating the plunger.

14. The uterine collection device of claim 1, further comprising a stop provided at the proximal end of the sheath for stopping the displacement of the plunger.

15. The uterine collection device of claim 3, wherein the sheath further includes a second lateral orifice a the proximate end and the stop includes a port substantially aligned with the second lateral orifice suitable for receiving one of a syringe and a stopper.

16. The uterine collection device of claim 15 further including a syringe suitable for insertion into the port of the stop for providing additional pressure at the distal lateral orifice.

17. The uterine collection device of claim 15 further including a stopper suitable for insertion into the port of the stop.

18. The uterine collection device of claim 1, wherein the sheath is made of transparent propylene.

19. The uterine collection device of claim 1, wherein the plunger is made of ethelyne-vinyl acetate.

20. The uterine collection device of claim 13, wherein the handle is made of a flexible acetyl resin.

21. The uterine collection device of claim 1, wherein the sheath includes at least one marking indicative of a reference length suitable for at least one manipulation.

22. A method of use of a uterine collection device having a generally tubular elongate shape extending from a proximal end suitable for manipulation to a distal end suitable for material collection, the method including the steps of:
providing a plunger extending from the proximal end to the distal end;
providing a sheath extending from the proximal end to the distal end having a transverse opening at the proximal end suitable for receiving the plunger, the distal end of the sheath forming a substantially closed transverse end, the closed transverse end of the sheath including a lateral orifice suitable for material collection through cause of displacement of the plunger within the sheath; and
providing at least one of the sheath and plunger with at least three portions with progressively decreasing radii of curvature extending from the proximal end to the distal end.

23. The method of claim 22, further comprising the step of providing at least one marking on the sheath indicative of a reference length suitable for at least one manipulation.

24. The method of claim 23, further comprising the steps of:
inserting the uterine collection device into one of the vaginal canal and the uterine canal until such point that the movement of the distal end of the uterine collection device is frustrated; and
rotating the uterine collection device to a point where the movement of the distal end of the uterine collection device is no longer frustrated.

25. The method of claim 22, further comprising the step of providing a port on the uterine collection device proximate the proximal end for receiving one of a stopper and a syringe.

26. The method of claim 25, further comprising the step of using a syringe with the port for one of collecting material and flushing the uterine collection device.
